(19) United States
(12) Patent Application Publication
(10) Pub. No.: US 2006/0041166 A1
(43) Pub. Date: Feb. 23, 2006

(54) Process for the continuous preparation of organic monoisocyanates and polyisocyanates

(76) Inventor: Joseph Y. Stuart, Baton Rouge, LA (US)

Correspondence Address: Warner J. Delaune, Jr.;Adams and Reese LLP, Suite 1900, 450 Laurel Street, Baton Rouge, LA, 70801, US

(21) Appl. No.: 10/923,561

(22) Filed: Aug. 20, 2004

(51) Int. Cl. C07C 263/10

(52) U.S. Cl. 560/348

(57) ABSTRACT

The invention relates to a process for the continuous preparation of organic isocyanates through the reaction of organic amines with phosgene in the presence of organic solvents under pressure whereby a concentrated phosgene-containing stream is mixed preferentially with an amine-containing stream in a jet mixer to create a combined jet of reacting amine-phosgene mixture, whereby the combined jet is discharged directly into a reactor vessel and the reactor vessel is operated at a temperature above the decomposition temperature of intermediate carbamoyl chloride products which can be formed upon mixing the aforementioned streams, wherein the combined jet is not pre-mixed with bulk reactor contents, wherein the jet mixer provides sufficiently rapid and thorough mixing and thereby enables an initial reaction temperature lower than the bulk reactor vessel temperature, and the combined jet entering the reactor has sufficient momentum to cause entrainment into it of a sufficient quantity of the bulk reactor contents to be rapidly dispersed and reach the bulk reactor temperature.

### BACKGROUND OF THE INVENTION

I. Field of the Invention

This invention pertains to a continuous process for the preparation of organic isocyanates.

II. Description of the Prior Art

The manufacture of organic mono-, di-, or polyisocyanates from the corresponding primary amines and phosgene is well known. Depending on the nature of the amines, the reaction is carried out either in the gas phase or the liquid phase, either batchwise or by means of a continuous process (W. Siefken, Liebigs Ann. 562, 75 (1949)); H. Ulrich, “Chemistry and Technology of Isocyanates”, John Wiley & Sons, Chichester, England, 1996; Ullmann's Encyclopedia of Industrial Chemistry, 7^{th} Edition, Volume A14, John Wiley & Sons, New York, 2003).

Organic isocyanates are now produced on a large industrial scale, usually in continuous liquid phase processes, where even small improvements in process efficiencies have significant economic importance. However, the conventional processes suffer from numerous disadvantages.

A. Two Stage Processes

The most frequently described processes for organic isocyanate production are two-stage processes, where amine, usually dissolved in organic solvent, and a stoichiometric excess of phosgene, sometimes also dissolved in organic solvent, are mixed in a first “cold” stage to ensure efficient reaction and minimization of by-products which affect both yield and quality. Intermediate amine hydrochlorides and carbamoyl chlorides are formed, and the reaction mixture is then fed to a second “hot” stage where the amine hydrochlorides are converted to carbamoyl chlorides and the carbamoyl chlorides are dissociated into isocyanate and hydrogen chloride. Optionally additional phosgene is added in this second stage. An early example of this type of process is described in U.S. Pat. No. 2,680,127.

Although the term “cold phosgenation” is often used for the first step, the temperature may be as high as 90 deg C (U.S. Pat. No. 2,908,703). The reaction of amines with phosgene is extremely fast, often described as “substantially” or “almost” instantaneous (U.S. Pat. Nos. 2,822,373; 3,287,387; 4,289,732). Specifically, U.S. Pat. No. 3,321,283 states that the reaction half life is 0.005 to 0.1 second and provides evidence in examples that the half life is of the order of 0.01 second. This means that substantially all of the heat of the first stage reaction is evolved in the first step, and the first stage reactors generally reach a temperature of 40-100 deg C, more usually 65-80 deg C (U.S. Pat. No. 3,188,337; 3,321,283; 3,947,484; 4,851,570; 5,117,048).

It is generally taught that the second stage is operated above the decomposition temperature of the carbamoyl chlorides, usually in the range 85 to 200 deg C, depending upon the type of isocyanate being produced. U.S. Pat. No. 3,781,320 teaches that the preferred temperature range for aromatic isocyanates such as toluene diisocyanate is 102-130 deg C, whereas for aliphatic isocyanates such as 4,4′-bis(diisocyantocyclohexyl)methane a temperature range of 150-175 deg C is preferred. Higher temperatures can be used but are not required.

These two-stage processes generally suffer from the disadvantage that a significant amount of solids are formed in the first stage. The presence of solids results in viscous fluids which make rapid mixing difficult, as well as the formation of blockages in the first reactor or transfer piping to the second reactor. One patent (U.S. Pat. No. 4,422,976) attempts to partially overcome this disadvantage by operating the first stage in a temperature and pressure regime where 30-70% of the intermediate carbamoyl chloride is dissociated to isocyanate. This gives a more fluid reaction mixture but there are still difficulties in transferring the reaction mixture containing solids to the second stage reactor. Two-stage processes also have long reaction times which require large, expensive reactors. The larger reactor volumes mean that the total inventory of phosgene in the equipment, which could potentially be released in the event of an equipment failure or other accident, is much larger. The increased hazard associated with a large inventory of phosgene restricts where a plant may be sited, and in order to reduce risk may lead to the need to apply secondary containment to the equipment, which is expensive, especially in view of the larger size of the equipment which must be so contained.

B. Single Stage Processes

Single stage processes are also described in the prior art, for example U.S. Pat. Nos. 2,683,160; 2,822,373; and 3,287,387. In single stage processes, the reactor or reaction system in which the amine, phosgene and reaction mixture are mixed together is operated at a temperature above the decomposition temperature of the carbamoyl chlorides. This type of process suffers from the disadvantage that the amine solution is added to a reaction mixture containing a higher concentration of free isocyanate, causing the formation of larger amounts of urea by-products. This must be counteracted by operating with a higher molar excess of phosgene over amine, often necessitating the use of high pressure. The excess phosgene must be recovered and recycled, which is expensive, and handling phosgene safely at high pressures requires more expensive equipment. The higher pressures may also lead to a higher quantifiable risk assessment rating from environmental authorities, which may restrict where a plant may be sited and lead to the need to apply expensive secondary containment to much of the equipment.

There are described both two-stage and single-stage process designs which incorporate recycling of the reaction mixture with amine addition into the recycle stream (U.S. Pat. Nos. 2,822,373; 3,465,021; 3,544,611; 4,128,569; 4,581,570; 5,599,968). These processes have the advantage that when the recycle rate is many times the amine feed rate, the effective stoichiometric excess of phosgene at the mixing point is much larger than the excess as measured by the ratio of the feed streams. However, they have the corresponding disadvantage that the added amine can react with an effectively higher concentration of isocyanate in the recycled reaction mixture, reducing the isocyanate yield and/or reducing the purity of the derived isocyanate. Many of the prior art processes have the further disadvantage that external pumped loops are used for rapid recirculation and these present a hazard with regard to handling phosgene solutions under pressure, requiring more expensive equipment. Again this may lead to a higher quantifiable risk assessment rating from environmental authorities, which may restrict where a plant may be sited and lead to the need to apply expensive secondary containment to much of the equipment.

Many prior art patents attempt to improve the yield in phosgenation processes and the quality of the isocyanate by the use of specialized high-speed mixing devices. Mechanical mixing devices may be used, for example high shear mixers (U.S. Pat. Nos. 3,321,283; 3,781,320), single stage pumps, turbomixers and colloid mills (all in U.S. Pat. No. 3,188,337), multiple stage pumps (U.S. Pat. No. 3,947,484), and rotor/stator mixers (U.S. Pat. No. 4,851,571). Static mixers are also described, for example tubular reactors with high turbulence (U.S. Pat. No. 3,226,410), venturi mixers (U.S. Pat. Nos. 3,507,626; 5,117,048), annular nozzles with opposing swirls (French patent no. 2,325,637) extremely fine smooth jet nozzles (U.S. Pat. No. 4,419,295), drive-jet nozzle for recycle stream into reaction chamber (U.S. Pat. No. 4,128,569), and fan jet nozzles (U.S. Pat. No. 4,289,295).

All of these prior art mixers either inject amine into a cold stage reactor, with the disadvantages of two-stage reactors outlined above, or inject amine into a hot stage reactor or the recycle stream from a hot stage reactor, with the disadvantages of needing higher phosgene excesses as described above, or have some residence time in the mixer or a pipe before being passed to either a cold or hot stage reactor, and during this residence time in the mixer or pipe there is the opportunity for blockages to form. As stated above, the first reaction is substantially instantaneous, so during this residence time in the mixer or pipe there is the opportunity for blockages to form due to the resulting solids.

The present invention overcomes the disadvantages described above by providing a brief mixing time for amine and phosgene in the absence of the bulk reaction mixture, permitting part of the reaction to take place below the main reactor temperature, and in the absence of that isocyanate which is present in the bulk reaction mixture. It thus retains many of the advantages of the “cold” stage of a two-stage reaction process. After this brief mixing time the jet of reacting components enters directly into the reactor and entrains reactor contents into it, thereby rapidly dispersing and taking advantage of the larger phosgene excess in the reactor bulk. The bulk reactor is operated above the decomposition temperature of the intermediate carbamoyl chlorides, but the design of the mixer largely prevents free amine coming into contact with isocyanate in the bulk, thereby minimizing urea formation and permitting operation at lower, more economical, overall stoichiometric excesses of phosgene than is possible in prior art single stage processes. The mixer jet enters directly into the bulk reactor, so there is no intermediate vessel or pipework which can become blocked by reaction solids.

Many designs have been described for equipment to provide residence time at the hot stage of phosgenation. These include stirred tanks (U.S. Pat. Nos. 3,287,387, 4,422,976), vertical tube reactors (U.S. Pat. No. 3,188,337), packed columns (U.S. Pat. No. 3,829,458), perforated plate columns (U.S. Pat. No. 4,851,570), distillation columns (U.S. Pat. No. 3,544,611), or valve tray columns or bubble cap tray columns with relatively high liquid weirs (U.S. Pat. No. 6,576,788). Some designs for hot stage phosgenation incorporate recirculation circuits. These may be external pumped loops (U.S. Pat. Nos. 3,781,320; 3,829,458; 4,128,569), or natural circulation systems either internal or external to the reactor (U.S. Pat. No. 4,581,174). U.S. Pat. Nos. 3,465,021 and 5,599,968 operate natural recirculation systems for “cold” stage phosgenation (up to 100 deg C) but then require further hot stage finishing reactors.

Most designs incorporate some means of separation of the gases generated in the hot stage, either integrally with the hot stage reactor (U.S. Pat. Nos. 3,781,320, 4,422,976) or in a gas-liquid separator subsequent to the residence time apparatus (U.S. Pat. Nos. 3,287,387; 3,829,458).

The jet mixer of the present invention can be connected directly to many of the above mentioned designs of reactor vessels and will show advantages, provided that they are operated in the temperature range and pressure range of the present invention. However in the preferred embodiment a single bulk reactor incorporates both an internal circulation path and gas-liquid disengagement.

### SUMMARY OF THE INVENTION

Therefore, one object of the present invention is to provide a process for the continuous preparation of organic isocyanates which reduces equipment costs by enabling operation within a single reactor vessel.

Another object of the present invention is to provide a process for the continuous-preparation of organic isocyanates which uses a single stage process.

A further object of the present invention is to provide a process for the continuous preparation of organic isocyanates which allows a brief mixing time for amine and phosgene in the absence of the bulk reaction contents.

Yet another object of the present invention is to provide a process for the continuous preparation of organic isocyanates which minimizes urea formation and permits operation at lower, more economical overall stoichiometric excesses of phosgene.

Accordingly, a process for the continuous preparation of organic isocyanates through the reaction of organic amines with phosgene in the presence of organic solvents under pressure is provided, comprising the steps of mixing a phosgene-containing stream with an amine-containing stream in a jet mixer to create a combined jet of reacting amine-phosgene mixture; discharging the combined jet from the jet mixer directly into a reactor vessel containing bulk reactor contents; and operating the reactor vessel at a vessel temperature above the decomposition temperature of intermediate carbamoyl chloride products formed in the course of the reaction. In the foregoing process, the combined jet is not pre-mixed with the bulk reactor contents. The jet mixer advantageously provides sufficiently rapid and thorough mixing to enable an initial reaction temperature of the reacting amine-phosgene mixture lower than the vessel temperature, and the discharge of the combined jet entering the reactor vessel has sufficient momentum to cause entrainment into the combined jet of a sufficient quantity of the bulk reactor contents to be rapidly dispersed and reach the vessel temperature.

### BRIEF DESCRIPTION OF THE DRAWINGS

FIG. 1 is a preferred embodiment of an organic isocyanate reaction system having an internal and external heat source for maintaining the reactor vessel temperature.

FIG. 2 is an alternative embodiment of the reaction system of FIG. 1 including an internal heat exchanger and draft tube.

### DETAILED DESCRIPTION OF THE PREFERRED EMBODIMENTS

The process according to the invention is quite generally applicable to the manufacture of organic isocyanates which can be obtained by reacting amines with phosgene. For example, monoisocyanates, diisocyanates and/or polyisocyanates can be manufactured from the corresponding organic monoamines, diamines and polyamines.

Suitable organic monoamino compounds have the formula R—NH₂, where R is an unsubstituted or substituted monofunctional aliphatic, cycloaliphatic or, preferably, aromatic radical having 1 to 20, preferably 6 to 12, carbon atoms. Examples are aliphatic monoamines, e.g., methylamine, ethylamine, butylamine, octylamine and stearylamine, cycloaliphatic monoamines, e.g., cyclohexylamine, and especially aromatic monoamines, e.g., aniline, toluidines, naphthylamines, chloroanilines and anisidines.

Preferably, however, the diisocyanates and polyisocyanates, which are of importance for the industrial manufacture of polyurethanes, are manufactured from the corresponding diamines and polyamines by the new process. Suitable diamino compounds have the formula H₂N—R′—NH₂, where R′ is a difunctional aliphatic or cycloaliphatic radical having 2 to 18, preferably 4 to 12 carbon atoms or, preferably, is a functional aromatic radical which consists of one or more aromatic rings having from 6 to 18 carbon atoms directly linked to one another or linked via divalent bridge members, e.g., —O—, —SO₂—, —CH₂— and —C(CH₃)₂—. The diamino compounds and/or polyamino compounds may be used individually or as mixture.

Said aliphatic, cycloaliphatic, or, preferably, aromatic diamino compounds are, for example: 1,4-diaminobutane, 1,6-diaminohexane, 1,10-diaminodecane, 1,12-diaminododecane, 1,4- or 1,3-diaminocyclohexane, 4,4′-diaminodicyclohexyl, 4,4′-, 2,4′-, 2,2′-diaminodicyclohexylmethane, 3,3′-dimethyl-4,4′-diaminodicyclohexylmethane, 4,4′-diaminodiphenyl-, 1,4- or 1,3-diphenylenediamine, 1,5- or 1,8-naphthylenediamine, 2,4- or 2,6-toluenediamine, and 2,2′-, 2,4′- or 4,4′-diaminodiphenylmethane.

Examples of suitable polyamines are tri(p-aminophenyl)methane, 2,4,6-triamino-toluene and condensation products which are obtained from substituted or unsubstituted aniline derivatives and aldehydes or ketones in the presence of acids, e.g., polyphenyl-polymethylene-polyamines.

Preferable organic amines are: 1,6-hexamethylenediamine, mixtures of 1,6-hexamethylene, 2-methyl-1,5-pentamethylene, and 2-ethyl-1,4-butylenediamine, 3-aminomethyl-3,5,5-trimethylcyclohexylamine, 2,4′-, 4,4′-, 2,2′-diaminodiphenylmethane, or mixtures of at least two of the cited isomers, 2,4- and 2,6-toluenediamine or their mixtures, polyphenyl-polymethylene-polyamines or mixtures of diaminodiphenylmethanes and polyphenyl-polymethylenepolyamines.

The process according to the invention is particularly suitable for the manufacture of aromatic diisocyanates and/or polyisocyanates from the corresponding amines and is therefore preferentially used for this purpose.

Liquid phosgene is used as the other initial component. The liquid phosgene can be reacted as such or when diluted with a solvent suitable for phosgenation, for example, monochlorobenzene, dichlorobenzene, xylene, toluene, and the like. For purposes of the invention herein, references to a “phosgene-containing stream” means phosgene (which may or may not contain additional solvents) discharged by a jet mixer with an amine-containing stream, but which has not previously been mixed with the bulk reactor fluids.

Suitable inert organic solvents are compounds in which the amines and the phosgene are at least partially soluble. For example, chlorinated aromatic hydrocarbons, e.g., chlorobenzene, o-dichlorobenzene, p-dichlorobenzene, trichlorobenzenes, the corresponding chloro-toluenes and xylenes, chloroethylbenzene, monochlorodiphenyl, alpha.- and .beta.-naphthyl chloride, alkyl benzoates, and dialkyl phthalates, e.g., diethyl isophthalate, toluene and xylenes have proved particularly suitable. The solvents may be used individually or as mixtures. Advantageously, the solvent used has a lower boiling point than the isocyanate to be manufactured, so that the solvent can readily be separated from the isocyanate by distillation. The amount of solvent is advantageously such that the reaction mixture has an isocyanate content of from 2 to 40 percent by weight, preferably from 10 to 30 percent by weight, based on the total weight of the reaction mixture.

The amines may be used undiluted or as solutions in organic solvents. In particular, amine solutions with an amine content of from 5 to 50 percent by weight, preferably from 15 to 35 percent by weight, based on the total weight of the solution, are used.

The bulk reaction is advantageously carried out at from 100 Deg C to 200 Deg C, preferably from 120 Deg C to 180 Deg C, and at pressures of from 3 to 50 bars, preferably from 5 to 20 bars. The temperature used in the process according to the invention is above the decomposition point of the carbamoyl chloride formed as an intermediate product of the reaction of phosgene with amine. The only upper limits on the pressure are set by technical considerations and, at times, safety considerations, but higher pressures than those stated do not produce any further increase in yield. The rectification of the separated hydrogen chloride is, however, significantly easier at high pressure.

In order to perform one embodiment of the process of the invention, the bulk reaction mixture, which is comprised of a solvent, dissolved isocyanate, phosgene, hydrogen chloride, as well as phosgenation intermediates and by-products, is contained in a reactor. The reaction mixture may be recycled in a loop by means of a circulating pump or, preferably, by means of natural circulation. Natural circulation occurs where two columns of reaction mixture are connected by some means at both top and bottom, and the fluid in one column has a lower density than fluid in the other column, due to differences in temperature and/or gas content. The columns may be of any cross-sectional design but are preferably cylindrical. One column may be the reactor itself and the other an external column connected to the reactor near the upper and lower liquid levels, but preferably one column is the reactor and the other an internal column, for example a draft tube mounted with open ends, one near the bottom of the reactor, the other in the upper part of the reactor but under the normal liquid surface. The reactor may contain an agitator, but in the preferred embodiment the agitation provided by loop circulation is adequate. The reactor is heated, either by an external loop heater or, preferably, internal heaters. In order to minimize equipment, the heater may be integral with the construction of the draft tube. The feed amine, or preferably the feed amine solution, and the feed of phosgene or preferably phosgene solution are introduced into the reaction mixture through combined jet nozzles. The exit point of the combined jets is located directly in the reaction mixture, preferably in recycling reaction mixture. The jet mixer outlet may be positioned to aid the natural recirculation, since it will have directional momentum and gases will be generated in the vicinity of the outlet. The function of the jet mixer is important in achieving the most potential benefits of the invention. The phosgene solution jet must form a temporary shield around the amine solution jet, so that the central amine solution reacts with surrounding phosgene and cannot come into immediate contact with the bulk reaction mixture. This can be achieved with a coaxial jet mixer, or more preferably an impinging coaxial jet mixer with phosgene solution forming an annular jet around the amine solution. Most preferably, a coaxial jet mixer with protruding centerbody, as described in my co-pending US patent application number 20040008752, is used, whose disclosure is incorporated herein by reference. The advantage of this design is that the jets coalesce on the protruding centerbody and the mixing time is shortened. There are no pipes or surrounding cones or expansion chambers after the mixing point of the streams, so blockages cannot occur.

The velocity of each feed jet in the mixer is in the range of 2 to 50 meters per second, preferably 10 to 30 meters per second. As the combined jet enters the reaction mixture, its forward momentum causes bulk reactor contents to be entrained into it and it is rapidly dispersed. The temperature of the combined reacting jet approaches the adiabatic temperature due to reaction, but the temperatures of the feed streams are controlled so that this temperature is below the bulk reactor temperature, and will typically be below 100 deg C. As the combined jet is dispersed into the bulk reactor fluid, either directly into the reactor vessel or into a separate recirculating loop external to the reactor vessel and containing bulk reactor fluid, it rapidly attains the desired final reaction temperature. For purposes of the invention, it is not critical that the combined phosgene-amine jet be discharged directly into a reactor vessel per se, but into any vessel, external loop, or other conduit or containment means through which the bulk reactor fluids may be in circulation. It is to be understood that any of the foregoing containment means may conveniently be referred to herein as a “reactor vessel.” The brief period of lower reaction temperature in the presence of a molar excess of phosgene, but in the virtual absence of bulk reactor contents, in combination with the design and operation of the bulk reactor, leads to a more efficient overall phosgenation reaction. With respect to the technology of phosgenation, this improved efficiency can be exploited in a number of economically advantageous ways. First, at any particular phosgene to amine ratio of reactant concentrations, the isocyanate yield and/or quality will be improved. Alternatively the concentration of amine in the feed solution may be increased to give higher output at constant yield. Further in the alternative, the excess of phosgene used in the process, which is expensive to recycle, may be reduced. Of course, any combination of these alternatives may be chosen to give the most advantageous outcome.

Further efficiencies will be evident to those skilled in the art from the other design features of the preferred embodiment of the process. In the preferred embodiment, the bulk reactor contains a draft tube which promotes natural recirculation in an internal loop. The reactor is preferably of vertical cylindrical configuration. The amount of reaction mixture maintained in the circulating loop should be such that the volumetric ratio of the total amount of reaction mixture circulating in the loop relative to the amount of amine solution and phosgene solution added is from 100:1 to 1:1, preferably from 30:1 to 5:1. In order to achieve the desired recirculation rate, the vertical draft tube has a length of from 5 to 25 m, preferably from 10 to 15 m. The inner diameter of the tube and the diameter of the bulk reactor vessel will depend on the output requirements of the plant. A stream of the reaction mixture, which corresponds in volume to the total liquid charge, is removed from the bulk reactor or recirculation loop as a product solution in order to further process and isolate the isocyanate. The volume of the reactor plus optional loop can be set such that the mean residence times are adjustable from approximately one minute to four hours, preferably from five minutes to two hours.

In order to remove the hydrogen chloride released during phosgenation, a gas-liquid separation device is required. This may be a separate disengagement vessel subsequent to the bulk reactor, or in the preferred embodiment it is incorporated into the top of the bulk reactor, where the cylindrical section may be widened to improve disengagement. Phosgene in the off-gases is recovered for reuse in the reaction. Phosgene may be separated from hydrogen chloride by absorption in solvent or by rectification. In either case, the use of high pressures in the phosgenation reaction means that higher temperatures can be used for the gas separation, leading to cost savings on refrigeration.

It will be evident that the preferred reactor design minimizes equipment by eliminating as far as possible external loops, incorporating gas-liquid separation, and operating at high pressure. The reactor heater may be integral with the draft tube. The reactor volume is minimized by employing short reaction times at temperatures above the carbamoyl chloride decomposition temperature, so that it is feasible to use a single reactor. Furthermore, as suggested above, the volume of the reactor, including an optional loop can be established such that the mean residence times are adjustable from approximately one minute to four hours, and preferably from five minutes to two hours, based on the volumetric flow of the discharged product solution. As will be understood by those practicing the invention, the preferred range of five minutes to two hours for the mean residence time will depend upon the specific type of starting amine in the process, with aliphatic amines requiring longer to phosgenate to completion.

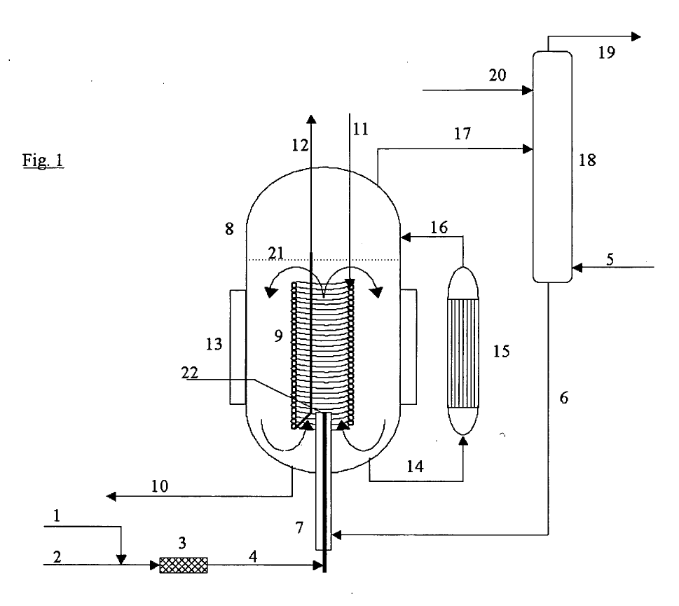

Having generally explained one or more preferred embodiments of the invention, the following description of exemplary operational systems is provided by reference to the figures. Referring specifically to FIG. 1, an amine stream is fed into the process at a controlled rate of flow through amine feed line 1. A solvent stream is fed into the process at a controlled rate of flow through solvent feed line 2. An amine-solvent solution is produced by mixing the amine stream with the solvent stream in amine and solvent mixer 3. Amine and solvent mixer 3 discharges into amine-solvent solution feed line 4 which discharges into jet mixer 7.

Fresh phosgene is fed into the process at a controlled rate of flow through fresh phosgene feed line 5. Fresh phosgene feed line 5 discharges into rectification system 18 and exits rectification system 18 combined with a recycle phosgene stream generated internally to rectification system 18. A predominantly phosgene stream containing some solvent and low concentrations of hydrogen chloride is discharged from rectification system 18 through total phosgene feed line 6 which discharges into jet mixer 7. The streams contained by amine-solvent solution feed line 4 and total phosgene feed line 6 flow through jet mixer 7 to discharge end 22 where they are intimately and rapidly mixed prior to dispersal into bulk reactor contents contained in reactor 8. Jet mixer 7 is preferably a coaxial jet mixer with a protruding centerbody as described in US patent application, publication number 20040008752, the disclosure of which is incorporated herein by reference. The streams contained by amine-solvent solution feed line 4 and total phosgene feed line 6 flow through jet mixer 7 to discharge end 22 mix and preferentially coalesce upon the protruding centerbody.

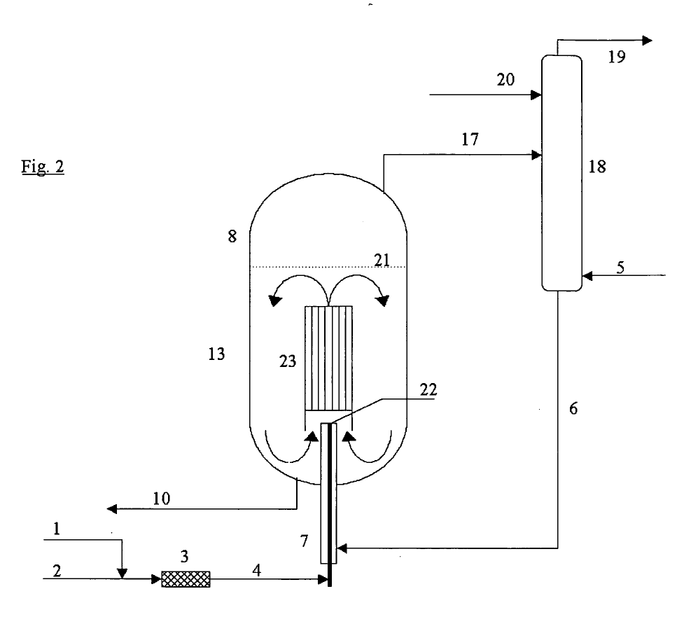

Discharge end 22 of jet mixer 7 is positioned beneath liquid surface 21 of reactor 8. Hydrogen chloride gas formed by the reaction of phosgene and amine, and the momentum of the discharge jet from jet mixer 7, create a natural circulation pattern in reactor 8. The natural circulation pattern is enhanced by the orientation and location of the discharge end 22 of jet mixer 7. A draft tube is used to improve natural circulation. The draft tube may or may not be combined with a heating apparatus. FIG. 1 shows draft tube and heating coil combined apparatus 9 where a simple draft tube would otherwise be installed. In the case of combined apparatus 9, a heating media or working fluid inlet 11 and outlet 12 are also present. Alternatively, FIG. 2 shows draft tube and internal heat exchanger combined apparatus 23. Two other potential heating apparatuses are shown in FIG. 1 including external heating jacket 13 and external heater 15. External heater 15 further includes a feed line 14 from reactor 8 and a discharge line 16 returning heated mixture to reactor 8. If external heater 15 is used, it preferably operates on a natural circulation principal, avoiding the need for a circulation pump. The selection of the particular configuration of heat exchange equipment is dependent upon the heating requirement of the particular amine being reacted, the scale of the process, and the geometry of the chosen process equipment.

Product solution discharge line 10 discharges a product and solvent solution from reactor 8 which is further processed by conventional means to isolate the product and purify the solvent for recycle in the process. Reactor vapor discharge line 17 carries hydrogen chloride, phosgene, and solvent vapors from reactor 8 to rectification system 18. Solvent feed line 20 optionally provides solvent from a solvent source (not shown) to assist in the rectification process. Rectification system 18 separates a nearly pure hydrogen chloride stream from the hydrogen chloride, phosgene, and solvent vapors from reactor 8. The nearly pure hydrogen chloride stream is discharged from the process through hydrogen chloride discharge line 19. Unreacted phosgene and solvent vapors carried in line 17 are mostly separated in rectification system 18 and discharged from rectification system 18 through total phosgene feed line 6. If solvent absorption is used in rectification system 18 to separate recycle phosgene from the nearly pure hydrogen chloride stream in hydrogen chloride discharge line 19, then the solvent used for absorption is also discharged from rectification system 18 through feed line for phosgene 6.

I claim:

1. A process for the continuous preparation of organic isocyanates through the reaction of organic amines with phosgene in the presence of organic solvents under pressure, comprising the steps of:
(a) mixing a phosgene-containing stream with an amine-containing stream in a jet mixer to create a combined jet of reacting amine-phosgene mixture;
(b) discharging said combined jet from the jet mixer directly into a reactor vessel containing bulk reactor contents;
(c) operating the reactor vessel at a vessel temperature above the decomposition temperature of intermediate carbamoyl chloride products formed in the course of the reaction;
wherein the combined jet is not pre-mixed with the bulk reactor contents, wherein the jet mixer provides sufficiently rapid and thorough mixing to enable an initial reaction temperature of the reacting amine-phosgene mixture lower than the vessel temperature, and wherein the discharge of the combined jet entering the reactor vessel has sufficient momentum to cause entrainment into the combined jet of a sufficient quantity of the bulk reactor contents to be rapidly dispersed and reach the vessel temperature.

2. The process of claim 1, wherein the phosgene-containing stream contains one or more solvents selected from the group consisting of chlorinated aromatic hydrocarbons, chlorobenzene, o-dichlorobenzene, p-dichlorobenzene, trichlorobenzenes, chloro-toluenes, xylenes, chloroethylbenzene, monochlorodiphenyl, alpha.- and .beta.-naphthyl chloride, alkyl benzoates, dialkyl phthalates, diethyl isophthalate, toluene and xylenes.

3. The process of claim 1, wherein the amine-containing stream contains one or more solvents selected from the group consisting of chlorinated aromatic hydrocarbons, chlorobenzene, o-dichlorobenzene, p-dichlorobenzene, trichlorobenzenes, chloro-toluenes, xylenes, chloroethylbenzene, monochlorodiphenyl, alpha.- and .beta.-naphthyl chloride, alkyl benzoates, dialkyl phthalates, diethyl isophthalate, toluene and xylenes.

4. The process of claim 1, wherein the bulk reactor contents are partially recirculated externally to the reactor vessel and the combined jet of reacting amine-phosgene mixture is discharged into the bulk reactor contents in recirculation.

5. The process of claim 1, wherein the bulk reactor contents are partially recirculated within the reactor vessel and the combined jet of reacting amine-phosgene mixture is discharged into the bulk reactor contents in recirculation.

6. The process of claim 1, wherein the jet mixer has at least a portion thereof residing with the reactor vessel, and wherein the reactor vessel further includes an internal circulation path and a liquid-gas disengagement device.

7. The process of claim 1, wherein the reactor vessel further includes a draft tube operatively positioned adjacent to the jet mixer in a manner to facilitate recirculation.

8. The process of claim 1, wherein the reactor vessel is internally heated.

9. The process of claim 1, wherein the reactor vessel operating temperature is between 100 degrees C and 200 degrees C.

10. The process of claim 1, wherein the organic amine in the amine-containing stream is selected from the group consisting of aliphatic, cycloaliphatic, aliphatic-aromatic, aromatic mono-, di- and polyamines.

11. The process of claim 1, wherein the organic amine in the amine-containing stream is selected from the group consisting of 1,6-hexamethylenediamine; mixtures of 1,6-hexamethylene-, 2-methyl-1,5-pentamethylene-, and 2-ethyl-1,4-butylenediamine; 3-aminomethyl-3,5,5-trimethylcyclohexylamine; 2,4′-, 4,4′-, 2,2′-diaminodiphenylmethane, and mixtures of at least two of the above-cited isomers: 2,4- and 2,6-toluenediamine and their mixtures; polyphenyl polymethylene polyamines; and mixtures of diaminodiphenylmethanes and polyphenyl polymethylene polyamines.

12. The process of claim 1, wherein the jet mixer utilizes impinging concentric annular jets to mix the amine-containing stream with the phosgene-containing stream.

13. The process of claim 12, wherein the phosgene-containing stream comprises an outer annular jet around the amine-containing stream.

14. The process of claim 12, wherein the velocities of the amine-containing stream and the phosgene-containing stream discharged from the jet mixer are between 2 and 50 meters/second.

15. The process of claim 12, wherein the concentric annular jets are briefly coalesced upon the surface of a protruding member of the jet mixer prior to dispersion of the coalesced stream into the reactor vessel.

16. The process of claim 12, wherein the concentric annular jets are briefly coalesced upon the surface of a protruding member of the jet mixer prior to dispersion of the coalesced stream into a recycle stream.

17. The process of claim 7, wherein the draft tube further includes an internal heating device within the reactor vessel.

18. The process of claim 7, where the number of reactors is one.
